(19) United States
(12) Patent Application Publication
(10) Pub. No.: US 2007/0031355 A1
(43) Pub. Date: Feb. 8, 2007

(54) Dermabrasion composition

(75) Inventor: Angeles Fonolla Moreno, Paris (FR)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/454,931

(22) Filed: Jun. 19, 2006

Related U.S. Application Data
(60) Provisional application No. 60/694,699, filed on Jun. 29, 2005.

(30) Foreign Application Priority Data
Jun. 22, 2005 (FR) 05 51714

(51) Int. Cl. A61K 8/26

(52) U.S. Cl. 424/59; 424/401

(57) ABSTRACT

A dermabrasion composition containing, in a cosmetically acceptable medium, aluminium oxide particles and pumice powder, and optionally a nonionic surfactant chosen from ethoxylated fatty acid esters that are solid at a temperature equal to 45° C., fatty acid esters of sugars, and mixtures thereof. The exfoliant composition can effectively attenuate the signs of ageing of the skin in the areas of the skin of the neckline and/or the hands and/or the arms, without causing any sensation of discomfort.

### REFERENCE TO PRIOR APPLICATIONS

This application claims priority to U.S. provisional application 60/694,699 filed Jun. 29, 2005, and to French patent application 0551714 filed Jun. 22, 2005, both incorporated herein by reference.

### FIELD OF THE INVENTION

The present invention relates to a cosmetic dermabrasion composition comprising aluminium oxide particles and pumice powder, and to its use for skincare.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Human skin has two compartments, namely a surface compartment, the epidermis, and a deep compartment, the dermis.

Natural human epidermis is composed mainly of three types of cells: the keratinocytes, which form the vast majority, the melanocytes and the Langerhans cells. Each of these cell types contributes, via its intrinsic functions, towards the essential role played by the skin in the body.

During the keratinization process, the keratinocytes located in the basal layer of the epidermis multiply and grow, thus pushing the older epidermal cells upwards and towards the surface of the epidermis. During this displacement, these cells become flattened and differentiate to form keratin. The superficial dead cells resulting from this keratinization process (corneocytes) constitute the horny layer of the epidermis, where they are separated by lipid layers and linked together via protein bonds (corneosomes) . These dead cells are gradually removed from the surface of the skin and replaced with new keratinized cells.

In young and healthy skin, the desquamation of the skin that takes place in this way is characterized by the removal of individual cells or of small lumps of cells. In contrast, with age or in the case of certain pathologies, the desquamation may be impaired, in the sense that an excess of keratinous material forms at the surface of the skin, resulting either in removal of the stratum corneum in the form of squamae (ageing of the skin, dry skin), or in an obstruction of the sebaceous follicles (acne).

Various agents for promoting desquamation, i.e. the removal of the “dead” cells lying at the surface of the horny layer of the epidermis, are known in the prior art.

It is known practice to use abrasive agents such as aluminium oxide in the form of corundum to exfoliate the surface of the skin (U.S. Pat. No. 6,290,976 and WO 02/072042). This exfoliation process is known as dermabrasion. Microdermabrasion is especially used by beauticians or dermatologists, who employ aluminium oxide microcrystals (in the form of corundum) to resurface the upper layers of the skin. These crystals are sprayed onto the skin using a machine that then sucks them off with the abraded skin.

However, for areas of skin that are particularly exposed, such as the neckline, the arms and the hands, the signs of ageing are more pronounced than those of the face, since these areas receive less daily protection against the UV radiation of light. Specifically, the daily application to the face of a day cream containing UV-screening agents that absorb UV rays attenuates the signs of ageing of the skin, for instance the appearance of wrinkles. On the other hand, the areas of skin mentioned above are rarely treated by the application of such a care cream and thus show more pronounced signs of ageing than those of the face.

Moreover, these specific areas of skin are areas that are highly touch sensitive. The exfoliant composition should therefore not create any sensation of discomfort such as heating, tautness, irritation, redness, stinging or drying-out of the skin.

It has been found that the application of an exfoliant composition comprising only corindum as abrasive particles does not satisfactorily attenuate the signs of ageing of the skin of these areas of skin, and in particular smooth out the wrinkles in these areas of skin. Furthermore, the abrasive property of corindum causes a certain amount of discomfort during the application of the exfoliant composition to these areas of sensitive skin.

### OBJECTS OF THE INVENTION

One object of the present invention is to provide an exfoliant composition that can effectively attenuate the signs of ageing of the skin of the areas of skin of the neckline and/or the hands and/or the arms and that does not create any sensation of discomfort during the application of the exfoliant composition to these areas of skin.

Another object of the invention is to provide an exfoliant composition that can effectively attenuate the visible or tactile irregularities of these areas of skin, and more particularly wrinkles. Thus attempts are made to create an effect of smoothing out the wrinkles of these areas of skin.

### SUMMARY OF THE INVENTION

The inventor has achieved these objects through the discovery of a composition combining pumice powder with aluminium oxide particles.

### DETAILED DESCRIPTION OF THE PREFERRED EMBODIMENTS

More specifically, one subject of the invention is a composition comprising, preferably in a cosmetically acceptable medium, aluminium oxide particles and pumice powder. Such a composition is also referred to as a dermabrasion composition.

A preferred dermabrasion composition according to the invention comprises aluminium oxide particles and pumice powder, in a cosmetically acceptable medium.

The term “cosmetically acceptable medium” means a medium that is compatible with the skin and/or its integuments, i.e. a medium that has an acceptable colour, odour and feel and that does not cause any unacceptable discomfort (stinging, tautness or redness) liable to put the user off using this composition.

Advantageously, the aluminium oxide particles have a purity of at least 95% and better still of at least 99%. Their mean particle size preferably ranges from 100 to 180 μm. Aluminium oxide is preferred for use in the present invention, in particular in anhydrous crystalline form (corindum).

Aluminium oxide particles that may be used herein include particles calcined at high temperature, until the crystalline structure of corindum (α-Al₂O₃) is obtained, and then treated to form grains having cutting edges and having a given particle size distribution, the particles preferably having a mean particle diameter of between 100 and 180 μm and preferably between 130 and 150 μm. Their distribution is advantageously such that none of the particles has a diameter of greater than 250 μm. Such particles are especially commercially available from the company MarkeTech International under the trade name Dermagrain. The particles referenced Dermagrain 900 are 99.55% pure crystalline alpha-alumina, with a mean particle diameter of about 140 μm, the particles all having a diameter of less than 250 μm. Less than 3% of the particles have a diameter of less than 105 μm. Other particles are available from the company Industrial Supply under the trade name ARL 100 and ARL 120. They are aluminium oxide particles with a mean particle size of 120 and 100 μm, respectively, and a particle size distribution ranging from 75 to 212 μm and from 63 to 180 μm, respectively.

The aluminium oxide particles may be present in the dermabrasion composition in any amount, including in a content ranging from 10% to 40% by weight and preferably ranging from 10% to 30% by total weight relative to the total weight of the composition.

The composition according to the invention also comprises pumice powder, in any amount, but advantageously in a content ranging from 0.5% to 10% by weight and preferably ranging from 1% to 5% by weight relative to the total weight of the composition.

The pumice powder is advantageously in the form of particles with a mean particle size of less than or equal to 200 μm, preferably ranging from 30 μm to 200 μm and preferentially ranging from 80 to 150 μm.

The composition may also comprise polyethylene powder, especially in a content ranging from 1% to 10% by weight and preferably ranging from 4% to 6% by weight relative to the total weight of the composition. Advantageously, the polyethylene powder may be in the form of particles with a mean size ranging from 5 to 300 μm and preferably ranging from 150 to 250 μm.

Advantageously, the composition may comprise one or more nonionic surfactants, for example one chosen from ethoxylated fatty acid esters that are solid at a temperature equal to 45° C., fatty acid esters of sugars, and mixtures thereof. These particular nonionic surfactants allow the composition to be rinsed off efficiently when it is applied to the skin, after having massaged the skin to effect microdermabrasion of the skin surface. More particularly, these nonionic surfactants allow, with water (especially cold water), the efficient removal of the aluminium oxide particles, the pumice and optionally the polyethylene powder present on the surface of the skin.

The ethoxylated fatty esters that are solid at a temperature of less than or equal to 45° C., which may be used in the composition according to the invention, are preferably esters formed from 1 to 100 ethylene oxide units and from at least one fatty acid chain containing from 16 to 22 carbon atoms. The fatty chain of the esters may be chosen especially from stearate, behenate, arachidate and palmitate units, and mixtures thereof. Examples of ethoxylated fatty esters that may be mentioned include stearic acid monoester comprising 8 ethylene oxide units (CTFA name: PEG-8 stearate), such as the product sold under the name Myrj 45 V by the company Uniqema, stearic acid monoester comprising 40 ethylene oxide units, such as the product sold under the name Myrj 52 (CTFA name: PEG-40 stearate) by the company ICI, behenic acid monoester comprising 8 ethylene oxide units (CTFA name: PEG-8 behenate), such as the product sold under the name Compritol HD5 ATO by the company Gattefosse. The stearic acid ester comprising 8 ethylene oxide units is preferably used.

The ethoxylated fatty ester that is solid at a temperature of less than or equal to 45° C. may prefereably be present in the composition according to the invention in a content ranging from 0.5% to 10% by weight, preferably ranging from 0.5% to 5% by weight and better still ranging from 1% to 4% by weight. relative to the total weight of the composition.

The fatty acid esters of sugars that may be used in the composition according to the invention are preferably solid at a temperature of less than or equal to 45° C. and may be chosen especially from esters and mixtures of esters of C₈-C₂₂ fatty acid and of sucrose, maltose, glucose or fructose, and esters or mixtures of esters of C₁₄-C₂₂ fatty acids and of methylglucose. These sugar esters may also be polyoxyethylenated or polyglycerolated, especially with 1 to 60 oxyethylene or polyglycerol units, and preferably polyoxyethylenated.

The C₈-C₂₂ or C₁₄-C₂₂ fatty acids forming the fatty unit of the esters that may be used in the composition of the invention comprise a saturated or unsaturated linear alkyl chain containing from 8 to 22 or from 14 to 22 carbon atoms, respectively. The fatty unit of the esters may be chosen especially from stearates, behenates, arachidonates, palmitates, myristates, laurates and caprates, and mixtures thereof. Stearates are preferably used.

Examples of esters or of mixtures of esters of fatty acid and of sucrose, maltose, glucose or fructose that may be mentioned include sucrose monostearate, sucrose distearate and sucrose tristearate, and mixtures thereof, such as the products sold by the company Croda under the names Crodesta F50, F70, F110 and F160 with an HLB (hydrophilic-lipophilic balance) of 5, 7, 11 and 16, respectively; and an example of esters or mixtures of esters of fatty acid and of methylglucose is polyglyceryl-3 methylglucose distearate, sold by the company Goldschmidt under the name Tegocare 450. Mention may also be made of glucose or maltose monoesters such as methyl o-hexadecanoyl-6-D-glucoside and o-hexadecanoyl-6-D-maltoside.

Polyoxyethylenated sugar esters that may be mentioned include polyethylene glycol ethers of methylglucose sesquistearate, for instance the product containing 20 ethylene oxide units (CTFA name: PEG-20 methylglucose sesquistearate), such as the product sold under the name Glucamate® SSE 20 by the company Chemron. This product is particularly preferred.

The sugar ester may be present in the composition according to the invention in any amount including in a content ranging from 1% to 10% by weight, preferably ranging from 1% to 6% by weight and better still ranging from 2% to 6% by weight relative to the total weight of the composition.

The nonionic surfactants may be present in the dermabrasion composition in any amount including in a content ranging from 0.2% to 12% by weight, preferably ranging from 0.2% to 8% by weight and preferentially ranging from 0.2% to 6% by weight relative to the total weight of the composition.

According to one particular embodiment of the invention, the composition of the invention may also contain one or more anionic surfactants. Their addition may further improve the stability of the composition.

Anionic surfactants that may be used in the composition include, and are preferably chosen from:
alkali metal salts of dicetyl and dimyristyl phosphate;
alkali metal salts of cholesteryl sulfate;
alkali metal salts of cholesteryl phosphate;
lipoamino acids and salts thereof such as monosodium and disodium acylglutamates, for instance the disodium salt of N-stearoyl-L-glutamic acid sold under the name Acylglutamate HS21 by the company Ajinomoto;
the sodium salts of phosphatidic acid;
phospholipids;
alkylsulfonic derivatives especially of formula (I): 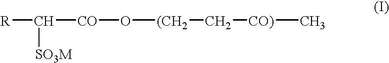
   in which R represents C₁₆-C₂₂ alkyl radicals, in particular C₁₆H₃₃ and C₁₈H₃₇ radicals taken as a mixture or separately and M is an alkali metal or alkaline-earth metal such as sodium;
   and mixtures thereof.

The anionic surfactant may be present in any amount including a content ranging from 0.01% to 6% by weight and more particularly from 0.5% to 3% by weight relative to the total weight of the composition.

The total content of nonionic and anionic surfactants may preferably range from 1% to 15% by weight and preferably from 3% to 10% by weight relative to the total weight of the composition.

Advantageously, the dermabrasion composition according to the invention is an emulsion, preferably an oil-in-water emulsion. The emulsion preferably comprises at least one oil.

The oils that may be used in the composition according to the invention are preferentially chosen from the group formed by:
oils of animal or plant origin, formed from fatty acid esters of polyols, in particular liquid triglycerides, for example sunflower oil, corn oil, soybean oil, avocado oil, jojoba oil, marrow oil, grapeseed oil, sesame seed oil, hazelnut oil, fish oils and glyceryl tricaprocaprylate, or plant or animal oils of formula R₉COOR₁₀ in which R₉ represents a higher fatty acid residue containing from 7 to 29 carbon atoms and R₁₀ represents a linear or branched hydrocarbon-based chain containing from 3 to 30 carbon atoms, in particular alkyl or alkenyl, for example purcellin oil or liquid jojoba wax;
natural or synthetic essential oils, for instance eucalyptus oil, lavandin oil, lavender oil, vetiver oil, litsea cubeba oil, lemon oil, sandalwood oil, rosemary oil, camomile oil, savory oil, nutmeg oil, cinnamon oil, hyssop oil, caraway oil, orange oil, geraniol oil, cade oil and bergamot oil;
synthetic oils such as parleam oil, polyolefins and liquid carboxylic acid esters;
mineral oils such as hexadecane, isohexadecane and liquid paraffin;
halogenated oils, especially fluorocarbons such as fluoroamines, for example perfluorotributylamine, fluorohydrocarbons, for example perfluorodecahydro-naphthalene, fluoro esters and fluoro ethers;
volatile or non-volatile silicone oils.

The polyolefins that may be used as synthetic oils are in particular poly-α-olefins and more particularly those of hydrogenated or non-hydrogenated polybutene type, and preferably hydrogenated or non-hydrogenated polyisobutene.

The liquid carboxylic acid esters that may be used as synthetic oils may be mono-, di-, tri- or tetracarboxylic acid esters. The total carbon number of the esters is generally greater than or equal to 10 and preferably less than 100 and more particularly less than 80. They are especially monoesters of saturated or unsaturated, linear or branched C₁-C₂₆ aliphatic acids and of saturated or unsaturated, linear or branched C₁-C₂₆ aliphatic alcohols, the total carbon number of the esters generally being greater than or equal to 10. Esters of C₄-C₂₂ di- or tricarboxylic acids and of C₁-C₂₂ alcohols and esters of mono-, di- or tricarboxylic acids and of C₂-C₂₈ di-, tri-, tetra- or pentahydroxylated alcohols may also be used.

Among the esters mentioned above, it is preferred to use alkyl palmitates such as ethyl palmitate, isopropyl palmitate, 2-ethylhexyl palmitate or 2-octyldecyl palmitate; alkyl myristates such as isopropyl myristate, butyl myristate, cetyl myristate or 2-octyldodecyl myristate; alkyl stearates such as hexyl stearate, butyl stearate and isobutyl stearate; alkyl malates such as dioctyl malate; alkyl laurates such as hexyl laurate and 2-hexyldecyl laurate; isononyl isononanoate; cetyl octanoate.

The composition according to the invention generally comprises an oily phase (oil and other fatty substances apart from surfactants) present in a content preferably ranging from 2% to 40% by weight, more particularly from 4% to 30% by weight and preferentially from 4% to 20% by weight relative to the total weight of the composition.

The composition according to the invention may comprise at least one thickener.

The thickeners can allow the viscosity of fluid compositions (5 cP) to be increased by at least a factor of 5, for a polymer concentration equal to 1% by weight. When added to a composition, they produce stable transparent compositions constituting milks or creams. The terms “milk” and “cream” mean compositions having a viscosity ranging from 0.5 to 150 poises (i.e. 0.05 Pa.s to 15 Pa.s) measured at 25° C. with a Rheomat 180 viscometer on spindle 3, 4 or 5 (depending on the viscosity range), at 200 s⁻¹.

The thickener may be chosen from polyethylene glycol esters or ethers containing from 80 to 350 ethylene oxide units; acrylic or methacrylic acid polymers; 2-acrylamido-2-methylpropanesulfonic acid polymers; acrylamide polymers; ethylene oxide polymers; polyvinyl alcohols; vinylpyrrolidone polymers; vinylcaprolactam polymers; polyvinyl methyl ether polymers; neutral acrylic polymers; C₁-C₂-alkyl-celluloses and derivatives thereof; C₁-C₃-alkyl guars or C₁-C₃-hydroxyalkyl guars; and mixtures thereof.

The thickener may be present in any amount including in a content ranging from 0.005% to 20%. by weight, preferably ranging from 0.01% to 10% by weight and preferentially ranging from 0.1% to 5% by weight relative to the total weight of the composition.

The dermabrasion composition may also comprise a heterogeneous polyholoside.

The polyholoside has the advantage of reducing the irritation resulting from the abrasive nature of aluminium oxide and also of complementing this abrasive, mechanical action by a “biological” exfoliant action, by inducing the release of corneocytes, as demonstrated in patent application EP-0 818 200.

According to the present invention, the term “heterogeneous polyholoside” means polymers consisting of a combination of different saccharides or of saccharides having the same empirical chemical formula but different geometrical configurations (for example D and L isomers).

These polymers differ both from polyheterosides, which consist of one or more saccharides and of a non-carbohydrate portion, and from homogeneous poly-holosides, which result from the combination of the same saccharide.

Thus, the heterogeneous polyholoside according to the invention consists solely of saccharides and results from the combination of at least two different saccharides.

The heterogeneous polyholosides may preferably consist of 2 to 10 saccharides, these compounds commonly being referred to as oligoholosides, or of more than 10 saccharides, these compounds commonly being referred to as polyholosides.

The saccharides present in the heterogeneous polyholoside may be chosen for example from any saccharide of natural or synthetic origin that may be envisaged, and especially such as:
aldoses, for instance
   pentoses: ribose, arabinose, xylose or apiose, for example,
   hexoses: glucose, fucose, mannose or galactose, for example,
ketoses such as fructose,
deoxyoses, such as rhamnose, digitoxose, cymarose or oleandrose,
saccharide derivatives such as uronic acids, for instance mannuronic, guluronic, galacturonic or glycuronic acids, or itols, for instance mannitol or sorbitol.

In the context of the present invention, a heterogeneous polyholoside alone or a mixture of heterogeneous polyholosides may be used.

The heterogeneous polyholoside may be branched or linear. It may also be substituted, for example with fatty chains, especially containing 8 to 30 carbon atoms.

Moreover, the heterogeneous polyholoside may be an alginate (poly mannuronate and guluronate) such as a sodium alginate, a propylene glycol alginate, a calcium alginate or a glyceryl alginate.

However, the heterogeneous polyholoside preferably comprises at least one fucose unit, which may be present in an amount of 10-90% by weight and preferably 15-35% by weight relative to the weight of polyholoside solids.

In particular, the heterogeneous polyholoside may comprise fucose, galactose and galacturonic acid units, and, for example, may comprise a linear chain of α-L-fucose, of α-D-galactose and of galacturonic acid. In this case, it preferably has a viscosity of 800-1200 mPa.s (Brookfield LV31 viscosity, 12 rpm, at 30° C.) when it is dissolved in water to a concentration of about 1% by weight. Such a polyholoside is especially available in the form of a 1% solution in water from the company Solabia under the trade name Fucogel 1000 PP

The heterogeneous polyholosides are preferably introduced into the composition in the form of an aqueous solution that may comprise 0.1% to 5% by weight of polyholoside.

The heterogeneous polyholoside may be present in the composition according to the invention in any amount including a content ranging from 0.0001% to 1% by weight and preferably from 0.0005% to 0.01% by weight relative to the total weight of the composition.

The dermabrasion composition according to the invention preferably has a pH generally of between 4 and 7.5 and preferably ranges from 5.5 to 6.5.

The composition of the invention may contain adjuvants and especially water-soluble or liposoluble active agents with cosmetic or dermatological activity. The liposoluble active agents are in the oily globules of the emulsion, whereas the water-soluble active agents are in the aqueous phase of the emulsion. Examples of active agents that may be mentioned include vitamins and derivatives thereof such as vitamin E and its esters such as vitamin E acetate, vitamin C and its esters, the B vitamins, vitamin A alcohol or retinol and its esters such as vitamin A palmitate, vitamin A acid or retinoic acid and its derivatives, provitamins such as panthenol, niacinamide, ergocalciferol, antioxidants, essential oils, humectants, sunscreens, moisturizers, proteins, ceramides and pseudoceramides, DHEA and its derivatives and biological precursors. Adjuvants that may also be mentioned include sequestrants, preserving agents, fillers, silicone gums, emollients, UV-screening agents, softeners, dyestuffs (pigments or dyes) and fragrances.

The amounts of these various adjuvants are not limited and include those conventionally used in the field under consideration, for example from 0.01% to 20% by weight relative to the total weight of the composition. These adjuvants and the concentrations thereof should be such that they do not significantly modify the desired property for the composition of the invention.

In addition, the composition according to the invention may be more or less fluid and may have the appearance of a white or coloured cream, a pomade, a milk, a lotion or a serum.

The composition according to the invention may be applied to the skin by manual massaging with the fingertips, or by mechanical massaging using a vibrating machine provided with a massaging head equipped with a pad, as described in patent application US 2001/0 046 506 or U.S. Pat. No. 6,652,888, for example. The composition is then removed by washing. To do this, the skin will generally be rinsed with water.

As a variant, the composition may be applied by spraying onto the skin using a machine that then sucks it off with the abraded skin.

The composition according to the invention effaces the visible and tactile irregularities of the surface of the skin in the area of the neckline and/or the arms (in particular the elbow area) and/or the hands (in particular the back of the hands), on account of its ability to mechanically abrade the surface of the skin.

A subject of the invention is thus also a process for caring for skin in the area of the neckline and/or the arms and/or the hands, comprising the application to the skin of a dermabrasion composition as described above.

This cosmetic treatment process is a type of exfoliation intended to resurface the upper layers of the skin, also known as microdermabrasion.

This process is intended in particular to attenuate the visual or tactile irregularities of the surface of the skin in the areas mentioned above, in particular to attenuate wrinkles and/or to smooth out the skin.

In particular, the treatment process comprises the successive application:
of a dermabrasion composition as described above, followed by its removal by washing, especially with cold water (room temperature);
of a cosmetic composition comprising at least one moisturizer, referred to as a moisturizing composition.

The frequency at which this product may be used will depend on the desired result. It will generally be used one to three times a week and preferably twice a week, for a period ranging from one week to six months and preferably from four to eight weeks.

This process is advantageously performed on individuals showing signs of ageing of the skin, such as wrinkles in the areas of skin of the neckline and/or the arms and/or the hands.

The moisturizer present in the moisturizing composition is not limited and may be chosen from ceramides, sphingoid-based compounds, lecithins, glycosphingolipids, phospholipids, cholesterol and its derivatives, phytosterols (stigmasterol, β-sitosterol or campesterol), essential fatty acis, 1,2-diacylglycerol, 4-chromanone, pentacyclic triterpenes such as ursoli- acid, petroleum jelly and lanolin; urea and its hydroxyalkyl derivatives, for instance hydroxyethylurea; threalose and its derivatives, hyaluronic acid and its derivatives, glycerol, pentanediol, sodium pidolate, serine, xylitol, sodium lactate, polyglyceryl acrylate, ectoin and its derivatives, chitosan, oligo- and polysaccharides, cyclic carbonates, N-lauroylpyrrolidonecarboxylic acid and N-α-benzoyl-L-arginine; steroidal derivatives (including DHEA, the 7-oxide and/or 17-alkyl derivatives thereof and sapogenins), methyl dihydrojasmonate, and vitamin D and its derivatives.

The moisturizer may be present in the moisturizing composition in a content ranging from 0.5% to 15% by weight and preferably ranging from 5% to 10% by weight relative to the total weight of the composition.

Advantageously, the moisturizing composition comprises at least one aqueous phase. In particular, it may be in the form of an emulsion and preferably in the form of an oil-in-water emulsion.

The moisturizing composition may comprise an oil, chosen especially from the oils described above.

The moisturizing composition may comprise any additive such as those described above, especially chosen from thickeners, surfactants, adjuvants and especially water-soluble or liposoluble active agents. The contents described above for the various additives also apply to the moisturizing composition.

A subject of the invention is also a kit comprising:
a first packaging containing a dermabrasion composition as described above,
a second packaging containing a moisturizing composition comprising at least one moisturizer as described above.

The first and second packaging may be a container delimiting at least one compartment, the said container being closed by means of a closing member.

The container may be in any suitable form. It may especially be in the form of a bottle, a tube, a jar, a case, a can, a sachet or a box.

The closing member may be in the form of a removable stopper, a lid, a cover, a tear-off strip or a cap, especially of the type comprising a body fixed to the container and a cap articulated on the body. It may also be in the form of a member ensuring the selective closure of the container, especially a pump, a valve or a clapper.

The composition may be contained directly in the container, or indirectly. By way of example, the composition may be arranged on an impregnated support, especially in the form of a wipe or a pad, and arranged (individually or in plurality) in a can or a sachet. Such a support incorporating the composition is described, for example, in patent application WO 01/03538.

The closing member may be coupled to the container by screwing. Alternatively, the coupling between the closing member and the container may take place other than by screwing, especially via a bayonet mechanism, by click-fastening, gripping, welding, bonding or magnetic attraction. The term “click-fastening” in particular means any system involving the passing of a rim or bead of material by elastic deformation of a portion, especially of the closing member, followed by return to the elastically unstressed position of the said portion after the rim or bead has been passed.

The container may be at least partly made of thermoplastic material. Examples of thermoplastic materials that may be mentioned include polypropylene and polyethylene.

Alternatively, the container is made of a non-thermoplastic material, especially of glass or metal (or alloy).

The container may have rigid walls or deformable walls, especially in the form of a tube or a tube bottle.

The container may comprise means for causing or facilitating the distribution of the composition. By way of example, the container may have deformable walls so as to make the composition come out in response to a positive pressure inside the container, this positive pressure being caused by elastic (or non-elastic) squeezing of the walls of the container.

The container may consist of a box with a base delimiting at least one housing containing the composition, and a lid, especially articulated on the base, which is capable of at least partly covering the said base. Such a box is described, for example, in patent application WO 03/018423 or in patent FR 2 791 042.

The kit described above may also contain a notice including indications regarding its mode of use, for the implementation of the process described above.

The content of the patents or patent applications mentioned previously are incorporated by reference into the present patent application.

The invention is illustrated in greater detail by the example described below, the contents being expressed as weight percentages.

### EXAMPLE 1

a) A microdermabrasion composition A comprising the ingredients below was prepared:

| | |
|---|---|
| Aluminium oxide (Dermaqrain 900 from the | 20% |
| company Marke Tech International) | |
| Pumice powder | 2.5% |
| Polyethylene powder | 4% |
| Shea butter | 1% |
| Liquid paraffin | 3% |
| Stearic acid | 1.2% |
| Oxyethylenated (20 EO) sorbitan monostearate | 0.5% |
| PEG-8 stearate (Myrj 45 V from the company | 1.5% |
| Uniqema) | |
| Diglyceryl distearate | 2% |
| PEG-20 ether of methylglucose sesquistearate | 3% |
| (Glucamate ® SSE 20 from the company Chemron) | |
| Polyacrylamidomethylpropanesulfonic acid | 2% |
| neutralized with aqueous ammonia and | |
| crosslinked (Hostacerin AMPS from the company | |
| Clariant) | |
| Polysaccharide at 1% in water (Fucogel 1000 | 1.7% |
| PP from the company Solabia) | |
| Glycerol | 7% |
| Preserving agents | qs |
| Octanediol | 0.4% |
| Water | qs 100% |

b) Moisturizing composition B:

An oil-in-water emulsion having the composition below was prepared:

| | |
|---|---|
| Soybean oil | 2% |
| Apricot oil | 1% |
| Shea butter | 1.5% |
| Monosodium salt of N-stearoylglutamic acid | 0.2% |
| Mixture of glyceryl mono, distearate and of | 0.5% |
| polyethylene glycol stearate (100 EO) | |
| (Arlacel 165 FL from the company Uniqema) | |
| Polydimethylsiloxane containing α,β-stearoxy | 1.5% |
| groups (Abil Wax 2434 from the company | |
| Goldschmidt) | |
| Cyclopentasiloxane | 5% |
| Dimethicone | 2% |
| Dimethiconol | 1.5% |
| Tocopheryl acetate | 0.5% |
| Fragrance | qs |
| Carbomer | 0.8% |
| Methylsilanetriol mannuronate at 1% in water | 1% |
| (Algisium C from the company Exsymol) | |
| Sodium hydroxide | 0.25% |
| Glycerol | 7% |
| Hydroxyproline | 1.5% |
| Preserving agents | qs |
| Propylene glycol | 3% |
| Water | qs 100% |

Each composition A and B is packaged separately in a tube in order thus to form a skincare kit.

The dermabrasion composition A was applied to the area of the neckline, the arms (in particular the elbow area) and the backs of the hands, these areas of skin being massaged for about 2 minutes. The composition was then removed with cold water. After drying the treated skin, the moisturizing composition B was applied.

On a panel of 42 women applying the treatment 3 times a week, it was found that after 2 weeks of treatment, the areas of the neckline, the arms and the hands showed perceptible smoothing-out of the skin wrinkles and also good softness of the skin. After 4 weeks of treatment, the results show higher efficacy as regards the smoothing-out of the wrinkles and the softness effect, compared with the results obtained after 2 weeks of treatment.

The above written description of the invention provides a manner and process of making and using it such that any person skilled in this art is enabled to make and use the same, this enablement being provided in particular for the subject matter of the appended claims, which make up a part of the original description and including a cosmetic composition comprising, in a cosmetically acceptable medium, aluminium oxide particles and pumice powder.

As used above, the phrases “selected from the group consisting of,” “chosen from,” and the like include mixtures of the specified materials. Terms such as “contain(s)” and the like as used herein are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition comprising, in a cosmetically acceptable medium, aluminium oxide particles and pumice powder.

2. The composition according to claim 1, wherein the oxide particles have a mean size ranging from 100 to 180 μm.

3. The composition according to claim 1, wherein the aluminium oxide is present in a content ranging from 10% to 40% by weight relative to the total weight of the composition.

4. The composition according to claim 1, wherein the pumice powder is in the form of particles with a mean particle size of less than or equal to 200 μm.

5. The composition according to claim 1, wherein the pumice powder is present in a content ranging from 0.5% to 10% by weight relative to the total weight of the composition.

6. The composition according to claim 1, further comprising polyethylene powder.

7. The composition according to claim 6, wherein the polyethylene powder is present in a content ranging from 1% to 10% by weight relative to the total weight of the composition.

8. The composition according to claim 1, further comprising a nonionic surfactant chosen from ethoxylated fatty acid esters that are solid at a temperature equal to 45° C., fatty acid esters of sugars, and mixtures thereof.

9. The composition according to claim 8, comprising an ethoxylated fatty acid ester that is solid at a temperature equal to 45° C. chosen from esters formed from 1 to 100 ethylene oxide units and from at least one fatty acid chain containing from 16 to 22 carbon atoms.

10. The composition according to claim 8, comprising at least one of a stearic acid monoester comprising 8 ethylene oxide units, a stearic acid monoester comprising 40 ethylene oxide units and a behenic acid monoester comprising 8 ethylene oxide units.

11. The composition according to claim 8, comprising an ethoxylated fatty ester that is solid at a temperature of less than or equal to 45° C. and which is present in a content ranging from 0.5% to 10% by weight relative to the total weight of the composition.

12. The composition according to claim 8, comprising a fatty acid ester of sugar chosen from esters and mixtures of esters of C₈-C₂₂ fatty acid and of sucrose, maltose, glucose or fructose, and esters or mixtures of esters of C₁₄-C₂₂ fatty acids and of methylglucose, these esters optionally being polyoxyethylenated or polyglycerolated.

13. The composition according to claim 12, comprising a fatty acid ester of sugar chosen from sucrose monostearate, sucrose distearate, sucrose tristearate, polyglyceryl-3 methylglucose distearate, methyl o-hexadecanoyl-6-D-glucoside, o-hexadecanoyl-6-D-maltoside, polyethylene glycol ethers of methylglucose sesquistearate, and the polyethylene glycol ether (20 EO) of methylglucose sesquistearate.

14. The composition according to claim 8, comprising sugar ester present in a content ranging from 1% to 10% by weight relative to the total weight of the composition.

15. The composition according to claim 8, wherein the nonionic surfactants are present in the composition in a content ranging from 0.2% to 12% by weight relative to the total weight of the composition.

16. The composition according to claim 1, further comprising an anionic surfactant.

17. The composition according to claim 16, wherein the anionic surfactant is chosen from:
alkali metal salts of dicetyl and dimyristyl phosphate;
alkali metal salts of cholesteryl sulfate;
alkali metal salts of cholesteryl phosphate;
lipoamino acids and salts thereof such as monosodium and disodium acylglutamates;
the sodium salts of phosphatidic acid;
phospholipids;
alkylsulfonic derivatives; and mixtures thereof.

18. The composition according to claim 17, wherein the anionic surfactant is present in a content ranging from 0.01% to 6% by weight relative to the total weight of the composition.

19. The composition according to claim 1, further comprising at least one surfactant chosen from non-ionic and ionic surfactants, and wherein the total content of nonionic and ionic surfactants ranges from 1% to 15% by weight relative to the total weight of the composition.

20. The composition according to claim 1, wherein the composition is an oil-in-water emulsion.

21. The composition according to Claim i, further comprising an oily phase present in a content ranging from 2% to 40% by weight relative to the total weight of the composition.

22. The composition according to claim 1, further comprising a thickener.

23. The composition according to claim 22, wherein the thickener is chosen from polyethylene glycol esters or ethers containing from 80 to 350 ethylene oxide units; acrylic or methacrylic acid polymers; 2-acrylamido-2-methylpropanesulfonic acid polymers; acrylamide polymers; ethylene oxide polymers; polyvinyl alcohols; vinylpyrrolidone polymers; vinylcaprolactam polymers; polyvinyl methyl ether polymers; neutral acrylic polymers; C₁-C₂-alkyl-celluloses and derivatives thereof; C₁-C₃-alkyl guars or C₁-C₃-hydroxyalkyl guars; and mixtures thereof.

24. The composition according to claim 23, wherein the thickener is present in a content ranging from 0.005% to 20% by weight relative to the total weight of the composition.

25. The composition according to claim 1, further comprising a heterogeneous polyholoside.

26. The composition according to claim 1, further comprising an alginate.

27. The composition according to claim 25, wherein the heterogeneous polyholoside comprises at least one fucose unit.

28. The composition according to claim 27, wherein the heterogeneous polyholoside comprises fucose, galactose and galacturonic acid units.

29. The composition according to claim 25, wherein the heterogeneous polyholoside is present in a content ranging from 0.0005% to 0.01% by weight relative to the total weight of the composition.

30. The composition according to claim 1, further comprising at least one additive chosen from vitamins, provitamins, antioxidants, essential oils, humectants, moisturizers, proteins, ceramides and pseudoceramides, DHEA and its derivatives and biological precursors, sequestrants, preserving agents, fillers, silicone gums, emollients, UV-screening agents, softeners, dyestuffs and fragrances.

31. A process for caring for skin in the area of the neckline and/or the arms and/or the hands, comprising applying to skin in the area of the neckline and/or the arms and/or the hands a composition according to claim 1.

32. The process according to claim 31, comprising application of a composition according to claim 1 to skin in the area of the neckline and/or the arms and/or the hands, followed by removal of the composition by washing, followed by the application to skin from which the composition has been removed of a moisturizing composition comprising at least one moisturizer.

33. The process according to claim 31, wherein the composition is applied to skin in need of attenuating the visual or tactile irregularities of the surface of the skin.

34. A kit comprising:
a first packaging comprising a composition according to claim 1,
a second packaging comprising a moisturizing composition comprising at least one moisturizer.
